(19) United States
(12) Patent Application Publication
(10) Pub. No.: US 2009/0240289 A1
(43) Pub. Date: Sep. 24, 2009

(54) Orthodontic Anchoring Screw

(76) Inventors: Holger Zipprich, Darmstadt (DE); Björn Ludwig, Trarben-Trarbach (DE)

Correspondence Address: KING & SPALDING, 1185 AVENUE OF THE AMERICAS, NEW YORK, NY, 10036-4003, US

(21) Appl. No.: 12/095,098

(22) PCT Filed: Nov. 27, 2006
(86) PCT No.: PCT/EP06/11358

§ 371 Date: Dec. 17, 2008

(30) Foreign Application Priority Data
Nov. 27, 2005 (DE) 10 2005 056 119.5
Oct. 25, 2006 (DE) 10 2006 051 082.8

(51) Int. Cl. A61B 17/86

(52) U.S. Cl. 606/305; 606/301

(57) ABSTRACT

An orthodontic anchorage screw (1, 1′, 1″) with a barrel provided with a thread (8) and formed onto a screw head (2) shall guarantee a particularly high and lasting strength after having been put in place in the jaw bone. For this purpose, according to the invention, the thread (8) is designed, viewed along the longitudinal axis of the threaded barrel (4), with at least one non-constant thread parameter.

The invention relates to an orthodontic anchorage screw with a barrel provided with a thread and formed onto a screw head.

One of the main therapeutical aims of orthodontics is the specific and controlled movement of teeth. Such a translatory or else rotatory movement of a tooth is only possible by exerting, to a suitable extent, a force upon the tooth, which is able to move the tooth relative to the jaw. This force is then exerted upon the tooth in question by means of suitable means of fixation for relatively long periods of time, until the tooth has taken the desired position and/or orientation in the oropharynx. The specific charging of a tooth with such a force selected in a suitable manner, for example through braces, wires, rubbers or other suitable force-exerting elements requires, however, a suitable support of the force-exerting element in the oropharynx.

Usually, such force-exerting elements are supported by another tooth, two teeth suitably positioned relative to each other being connected, for example, by a clamping element which is then prestressed with tensile stress. Such an approach has, however, the disadvantage that, due to the concept, two teeth are symmetrically charged with forces, whereas this is intended and desired for one of the teeth only. The specific repositioning of one tooth is, therefore, perforce, accompanied by a change of position of the other tooth, which is undesired or acceptable to a certain extent only.

To counteract that, an anchorage can alternatively be effected by means of single-piece temporary screws, so-called orthodontic anchorage screws, which are temporarily screwed into the jaw bone and are removed after the displacement of the tooth has been completed. Contrary to dental-implant materials, which are designed for being lastingly integrated into the jaw bone and growing in with long-term stability, such anchorage screws must, therefore, be inserted in such a way that it remains possible to remove them from the bone at a later date. On the other hand, it must necessarily be taken into account, when using such anchorage screws, that during the active therapy phase, i.e. during the introduction of force properly speaking into the tooth needing treatment, a reliable seat with high load-carrying ability of the respective anchorage screw provided as a counterpoint is indispensible.

In this context, it has to be borne in mind that the anchorage screw, after having been screwed in, possesses a strength in the bone which is based on the tensioning of the screw with the bone. This strength is also called “primary stability”. As, however, the bone reacts to mechanical stresses and is repositioned, this strength will first of all decrease, due to the repositioning, with increasing wearing time. As from the moment of colonization of the screw material by cells, in particular bone cells, on the other hand, an additional strength develops through the compound of the cells with the screw material. This is called “oseointegration”. The strength in the bone should be sufficient during the entire duration of insertion to serve as an abutment for displacing at least one tooth. As, however, in the present systems, the primary stability is clearly reduced before a sufficient compound with the bone develops, the strength may in an intermediate phase be clearly reduced in comparison with the primary stability. In this phase, the undesired fact may happen that the screw is no longer able to transmit the necessary forces and is lost or is pulled out of the bone.

The invention is, therefore, based on the task to provide an orthodontic anchorage screw of the above-mentioned type with which a particularly high and lasting strength can be achieved after insertion into the jaw bone.

This task is solved according to the invention by designing the thread of the anchorage screw with at least one non-constant thread parameter, viewed along the longitudinal axis of the threaded barrel.

The invention is based on the consideration that for the desired sufficiently high strength of the inserted anchorage screw during the entire period of treatment, one should take into account, as a basis for the design of the system, in particular that time interval prior to the finally developing oseointegration, in which the above-mentioned reduction in strength may occur. In this context, it should be taken into account that this reduction in strength is mainly a consequence of rearrangement or adaptation processes in the surrounding bone, in the manner of structural transformations, in reaction to the inserted screw. To avoid an excessive reduction in strength, these effects should, therefore, at least in part, be purposefully reduced, offering in addition a mechanical retention. This can be effected in particular by generating different conditions in different spatial areas, through which the overall strength can be increased. For this purpose, it is provided to design the thread in a suitably irregular manner along the longitudinal axis of the threaded barrel.

As thread parameters, i.e. as data particularly suitable for characterizing the thread, a multitude of parameters can be taken into consideration. A thread is usually defined in particular by the thread parameters of thread pitch and depth of thread. The thread pitch is defined as the change of position in longitudinal direction of the thread axis per revolution, so that the thread pitch can be considered as the measure by which a screw penetrates per revolution into a material. Another thread characteristic is the depth of thread. The depth of thread characterizes how deep the thread geometry is cut into the material, thus indicating a measure for the profile depth of the thread. The larger the depth of thread, the smaller the root of the thread of the bolt or screw. Advantageously, thread pitch and/or thread profile are provided as the thread parameters which are designed in a non-constant manner, viewed along the longitudinal axis of the threaded barrel.

In view of the desired increase in strength, in particular an irregularly designed thread pitch, through which suitable tensions in the bone tissue, having a strength-increasing effect, can be induced, is advantageous. In particular, the bone, into which the temporary anchorage screw is screwed, is either compressed or stretched in screwing-in direction by the thread. The resulting additional tensioning of the bone with the screw increases the contact pressure between the temporary screw and the bone, which has an immediate effect on the strength.

Through the advantageously provided alternative or additional variation of the thread profile as a non-constant thread parameter, it is achieved that the bone in the screw channel, which is pre-cut or cut by the screw itself, is irregularly compressed, viewed in longitudinal direction of the threaded barrel. As a result, one obtains in the bone undercuts relative to the temporarily inserted screw, which increase the retention and counteract a rotatory movement.

The cross-section of the orthodontic anchorage screw, in particular in the area of its threaded barrel, can be, in the manner of common screw types, rotationally or cylindrically symmetrical and, thus, almost circular. In a particularly advantageous embodiment, however, the area of the cross-section of the threaded barrel is also specifically adapted to the design objective of high strength after insertion of the screw. Expediently, one makes use of the knowledge that after a certain period of time after insertion of the screw, a restructuring process will take place in the bone, during which the bone adapts itself to the contour of the inserted screw, the clamping effect originally fixing the screw in the surrounding bone material decreasing more and more. In this state, a rotationally symmetrical embodiment of the threaded barrel with almost circular cross-section will offer at best only little resistance to a rotatory movement of the screw, which might finally lead to a detachment of the screw. Clearly higher strengths can be achieved in this phase by advantageously designing the screw in the area of its threaded barrel for a rotation inhibition through positive locking with the surrounding bone material. This can be achieved by designing the threaded barrel in a suitably asymmetrical manner regarding the rotation about its longitudinal axis. Particularly advantageously, the cross-section of the threaded barrel is for that purpose of approximately triangular or trioval shape. Therefore, such an embodiment corresponds to the case that the non-constant thread parameter is the distance of the screw channel from the central axis of the threaded barrel.

Another possible non-constant thread parameter is, for example, an impression of structures or holes in the thread flanks. These can be worked into the thread flanks by suitable manufacturing processes, such as, for example, metal-cutting or forming techniques.

The variation of the non-constant thread parameter with the position along the longitudinal axis of the threaded barrel can be provided in particular continuously, for example linearly or following another function, non-recurrently or with sequential recurrence, with regular or irregular variation. In a particularly advantageous embodiment, however, the threaded barrel includes a number of main zones arranged, viewed along its longitudinal axis, one behind the other, in which the thread is designed in each case with almost constant thread parameters. Therefore, within one main zone, the thread and, thus, in this area also the surrounding bone tissue, has homogeneous properties. At the transition from one main zone to the adjacent main zone, the thread parameters provided in each case as being non-constant change discontinuously or discretely. Through suitable selection and dimensioning of the individual main zones, a particularly individual adaptation to the circumstances given in the jaw area can be achieved.

In a particularly advantageous embodiment, the threaded barrel includes two main zones, the thread in the first main zone being a single-start thread and the one in the second main zone adjacent thereto, being a two-start thread, with otherwise identical thread parameters, such as thread pitch and thread profile. The first main zone can be designed as a lower main zone, i.e. adjacent to the cutting point of the threaded barrel, and the second main zone, as an upper main zone, i.e. positioned between the first main zone and the screw head, particularly preferably with a barrel length of the second main zone of approximately 2 to 3 mm, as such a configuration can take into account in a particularly favorable manner the bone structure in the jaw, it being, furthermore, considered as particularly desirable to avoid by all means, in case of failure of the screw, a fracture of the screw—as opposed to the fact that the thread flanks are sheared off in the thread hole or on the screw or that the material of the thread hole expands so much that the thread slips through.

For this purpose, the overall system should, therefore, be designed in such a way that, in the manner of an integrated safety reserve, in case of doubt, the thread will fail in the bone before the screw fractures. Therefore, the thread of the screw should be adapted to a great extent to the bone/titanium material pairing. As all common self-cutting screw types will fracture in case of good bone quality before the thread in the bone fails, the thread should be purposefully weakened in the bone for the sake of the above-mentioned safety reserve, the functionality of the screw thread being achieved by making specific use of the anatomic conditions of the human jaw. The primary thread—in the area of the first main zone—is located, after insertion of the screw, in the bone area of the spongiosa. There, no risk of fracture of the screw exists, because the spongiosa is too soft. The secondary thread—in the area of the second main zone—, on the other hand, is adapted to the conditions of the compacta, which is situated on top of the spongiosa and has usually a thickness of 2 to 3 mm. The compacta has a relatively higher hardness, so that in this area, one must reckon with a higher stressing of the screw and risk of fracture. To counteract this, the thread is in this area purposefully weakened through the two-start design, as the bone substance is in particular weakened by the increased removal of material therefrom. That means that there are two thread types for two operations (secure screwing-in and increase of the resistance to fracture) and two bone types (spongiosa and compacta). The fact that the thread pitch is, nevertheless, maintained in both main zones, furthermore prevents additional tensions in the bone substance, so that the screw designed in such a way is particularly suited for application in this area.

The anchorage screw should be designed for a particularly easy handlability. To facilitate in this context the specific charging with a tightening torque and thus to particularly facilitate the insertion properly speaking of the screw, the outer contour of the screw head is advantageously designed as a polygonal contour, preferably an octagonal contour.

In a particularly advantageous embodiment, the orthodontic anchorage screw is designed for being applied together with fastening means usually applied in the therapy. These include, for example, braces, wires, spring and/or rubbers. Advantageously, the anchorage screw is, therefore, designed in such a way that the before-mentioned common fastening means can be fastened on the screw head without further efforts. These fastening means are in particular rectangular wires with a width of 0.018″ and 0.022″. For this purpose, the roof surface of the screw head is advantageously provided with a number of, preferably two, crossing, continuous profiled recesses. In another advantageous embodiment, the cross-section and/or the dimensioning of these recesses are adapted to a dental therapeutical brace element of the above-mentioned type. Thus, construction and function of the profiled recesses correspond to the so-called “slots” for such fastening means, applied, for example, with the so-called “brackets”, which are glued directly onto the teeth for dental treatment.

In another advantageous embodiment, the side face of the screw head is provided with a peripheral fastening groove, which can serve as an undercut for enabling a suitable fastening of the fastening rubbers for the wires or fastening means guided in the profiled recesses.

Advantageously, the orthodontic anchorage screw is made of titanium as the basic material.

The advantages achieved with the invention consist in particular in the fact that through the variing thread parameters locally different conditions in the bone mass, favoring the strength of the inserted anchorage screw, can purposefully be adjusted. The above-described configuration of the thread helps to increase the primary stability and/or to increase the strength through the oseointegration.

An exemplary embodiment of the invention is explained in detail by means of a drawing, in which:

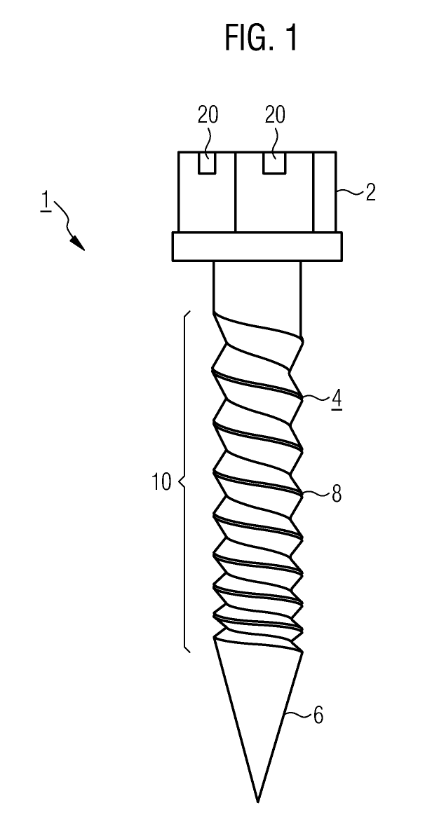

FIG. 1 to 3 each show an orthodontic anchorage screw,

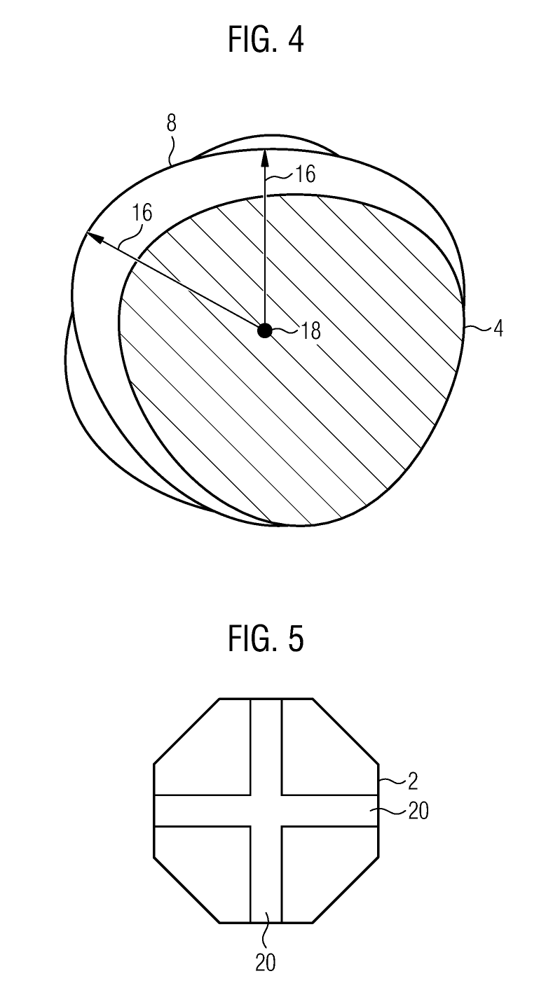

FIG. 4 shows the cross-section of the threaded barrel of the orthodontic anchorage screw, and

FIG. 5 is a top view of the screw head of the orthodontic anchorage screw.

Identical parts are identified by the same reference numbers in all figures.

The orthodontic anchorage screw 1 according to FIG. 1 is provided for temporary insertion into the jaw bone of a patient. The anchorage screw 1 shall serve, in the manner of an abutment, for a reliable fastening with high load-carrying ability of fastening elements or other force-exerting elements, through which, as part of the intended therapy, translatory or rotatory forces shall be exerted upon the tooth requiring treatment, so that the latter is displaced into a desired position. After successful completion of the treatment, the anchorage screw 1 can be removed from the jaw. In view of good compatibility with human tissue, the anchorage screw 1 is completely made of titanium.

The anchorage screw 1 comprises a threaded barrel 4, which is formed onto a screw head 2 and is provided above a cutting point 6 with a peripheral thread 8. The screw 1 can be designed in the manner of a countersunk screw, with which the cutting point 6 can directly be screwed into the unpretreated bone. Alternatively, it can, however, also be designed for use in pilot-drilled channels, the cutting point 6 being configured accordingly.

In view of the typical marginal conditions given for the intended orthodontic treatment, such as, for example, reliable load-carrying ability during a longer period, the anchorage screw 1 is particularly designed for a particularly reliable, particularly firm seating also on a medium-term basis, in the jaw bone after its insertion. The anchorage screw 1 is purposefully designed for reinforcing the achievable strength after insertion into the bone (the so-called primary strength) by purposefully tensioning the bone structure through the inserted screw and thus particularly increasing the strength. For this purpose, one or several of the thread parameters of the thread 8 are not kept constant, but vary as a function of the position, viewed along the longitudinal axis of the threaded barrel 4 or the threaded area 10 or the thread length.

The thread parameters to be taken into consideration are in particular the thread pitch, the thread profile, a surface contour on the thread (holes or grooves formed into the thread flanks) or the number of starts of the thread. The variation of the respective thread parameter can be continuously or non-continuously, i.e., for example, a continuous variation of the respective thread parameter can be configured, for example, linearly, according to a certain function, non-recurrently or recurrently, with regular or irregular variation, whereby essentially a continuous variation can take place along the longitudinal axis of the threaded barrel 4. Alternatively, however, it can also be provided, in the manner of discretely changing parameters, that the threaded barrel 4 includes a number of main zones 12 arranged, viewed in its longitudinal axis, one behind the other, thread parameters being kept constant within one main zone 12, and a discrete change of a thread parameter taking place at the transition from one main zone 12 to the adjacent main zone 12.

The exemplary embodiment according to FIG. 1 shows by way of example that the thread pitch of the thread 8, as a non-constant thread parameter, varies continuously in direction of the longitudinal axis of the threaded barrel 4. Through such an embodiment, i.e. through a variation of the thread pitch, viewed in longitudinal direction of the screw 1, it can be achieved that the bone into which the anchorage screw 1 is screwed is either compressed or stretched by the thread 8 in screwing-in direction. This additional tensioning of the bone with the anchorage screw 1 increases the contact pressure between the anchorage screw 1 and the bone. The increase of the contact pressure between the anchorage screw 1 and the bone increases the moment of static friction and sliding friction between them required after the screwing-in for releasing the anchorage screw 1.

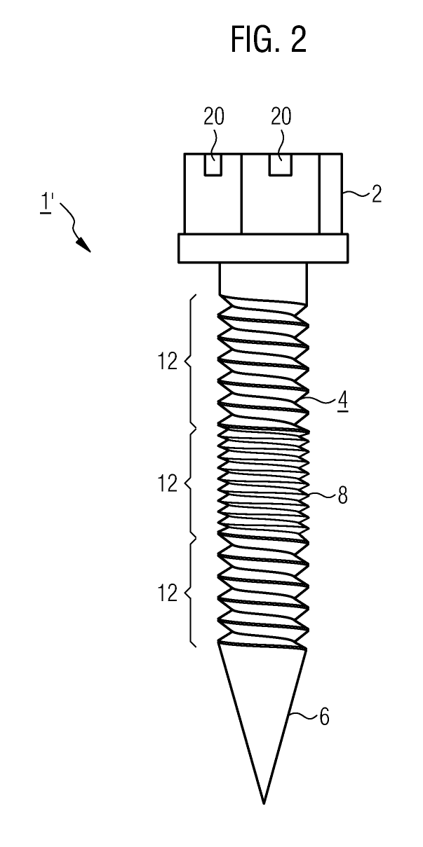

The exemplary embodiment according to FIG. 2, on the other hand, shows the case that the thread parameter is kept constant by segments and just changes discretely between adjacent segments or main zones 12 of the threaded barrel 4. It is evident from FIG. 2 that, viewed in longitudinal direction of the threaded barrel 4, a main zone 12 with a relatively clearly finer thread pitch is arranged between two main zones 12 with a relatively coarse thread pitch each.

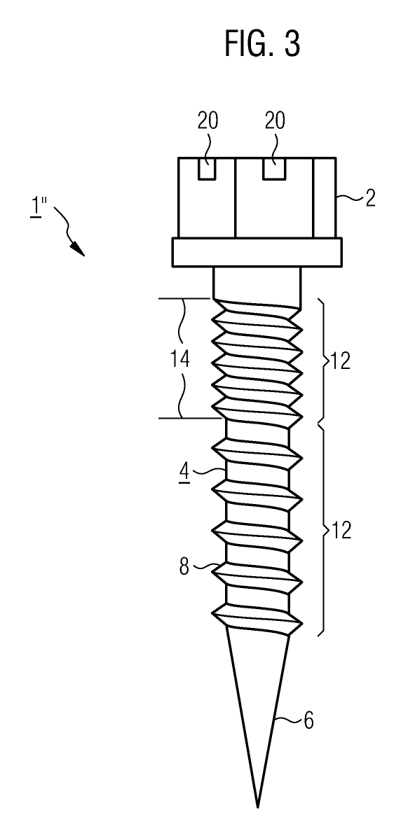

The exemplary embodiment according to FIG. 3 shows another orthodontic anchorage screw 1″, in which the threaded barrel 4 includes a plurality of—in the present case, two—main zones 12 in each of which the thread parameter is kept constant. In the example shown here, an upper main zone 12 is provided, having a thread 8 which, as compared with the other main zone 12 therebelow, has the same thread pitch and the same thread profile. These actually identical threads in the main zones 12 differ, however, by the fact that the thread 8 in the lower main zone 12 is configured as a single-start thread and the thread 8 in the upper main zone 12, as a two-start thread. With identical thread pitches, the thread 8 in the upper main zone 12 has, therefore, exactly double as many thread ribs per length unit as the thread 8 in the lower main zone 12, the upper main zone 12 having a length of approximately 2 to 3 mm.

The anchorage screw 1″ shown in the exemplary embodiment according to FIG. 3 is designed to a particularly high degree for an application in the jaw bone under observation of high safety measures, the design objective being based in particular on the fact that in case of failure of the anchorage screw 1, it must by all means be avoided that the screw fractures, so that the screw head 2 is torn off and the remaining screw body gets stuck in the jaw bone and would then have to be milled out. In order to purposefully and securely prevent this occurrence, the anchorage screw 1″ is specifically configured in such a way that in case of failure, instead of a fracture of the screw, it will not be the screw body that fails, but rather the thread or its anchorage in the bone. Accordingly, the thread contour and the formation of zones are specifically adapted to a typical layer arrangement of the bone in the area of the jaw. A human jaw includes a relatively hard upper layer, the so-called compacta, which has an average thickness of approximately 2 to 3 mm. Below it, there is the so-called spongiosa, a relatively softer bone zone. The anchorage screw 1″ according to FIG. 3 is specifically adapted to these conditions, because after the anchorage screw 1″ has been screwed in, the upper main zone 12 is situated in the jaw approximately in the area of the compacta, as indicated by the auxiliary lines 14, whereas the lower main zone 12 lies in the area of the spongiosa. As to the strength, the area in the spongiosa is a minor problem, as usually, when the anchorage screw 1″ is inserted, it does not offer sufficient strength to resist in case of doubt a load leading to a fracture of the screw. This could at best happen in the area of the compacta. In the area of the compacta, however, the density of the thread profile is double as high, due to the provided two thread starts, which finally weakens the grip and the strength of the thread as such in this area. It this way, it is specifically provided that the thread is sufficiently weakly dimensioned in this area, so that the thread in this bone area would yield before the screw fractures. Besides, the threads in the two main zones 12 have the same thread pitch, so that otherwise, the anchorage screw 1″ can be screwed in, in a particularly harmonious and smooth manner.

Alternatively or additionally, the thread parameter varying as a function of the position can, however, also consist in the fact that the distance of the thread profile from the main axis of the anchorage screw 1, 1′, 1″ varies as a function of the position. FIG. 4 shows an exemplary embodiment thereof, the cross-section of the threaded barrel 4 being represented in that case. The threaded barrel 4 has an approximately trioval cross-section, so that—as indicated by the arrows 16—, different distances of the thread profil from the screw axis 18 arise as a function of the position in the screw channel and, therefore, also as a function of the position in longitudinal direction of the anchorage screw 1, 1′, 1″. Besides, by such a triangular or trioval contour of the cross-section, it is also possible to achieve an increased strength of the seating of the anchorage screw 1, 1′, 1″ even after the adaptation processes in the bone tissue. This is possible because beyond the normal locking through static friction or sliding friction, a positive locking can also be achieved with such an embodiment, which will support the strength of the inserted screw even after the structural changes in the bone tissue have taken place.

It is evident from the top view of FIG. 5 that the screw head 2 is designed with an octagonal contour, in order to facilitate mounting and dismounting of the screw in the jaw using suitable tools. Furthermore, the roof surface of the screw head 2 is provided with two crossing, continuous profiled recesses 20. Their cross-section is rectangular and their dimensioning—a typical width of 0.018″ or 0.022″ being provided—is adapted to the fastening or adjusting elements, such as, for example, braces, wires, springs or rubbers, usually applied in orthodontic treatments. The profiled recesses 20 serve, therefore, for fastening the additional fastening means, in the manner of so-called slots or cross slots, so that a reliable force transmission to the tooth requiring treatment can be achieved, through an anchorage on the jaw. In this way, it can also be achieved, especially through the adaptation of the dimensioning or contouring of the profiled recesses 20 to the typical profiles and dimensions of these fastening means, in particular through the rectangular cross-section, that the desired forces can be transmitted in a particularly reliable manner and that both translatory and rotatory forces can be transmitted and fixed.

### LIST OF REFERENCE NUMBERS

1 Anchorage screw
2 Screw head
4 Threaded barrel
6 Cutting point
8 Thread
10 Threaded area
12 Main zone
14 Auxiliary line
16 Arrows
18 Screw axis
20 Profiled recess

1. Orthodontic anchorage screw (1, 1′, 1″) with a barrel (4) provided with a thread (8) and formed onto a screw head (2), which includes a number of main zones (12) arranged, viewed along its longitudinal axis, one behind the other, in each of which the thread (8) is designed with approximately constant thread parameters, the main zones (12) differing in at least one thread parameter, this thread parameter being the thread profile and/or the number of starts of the respective thread segment.

2. Orthodontic anchorage screw (1, 1′, 1″) according to claim 1, in which the main zones (12) differ additionally in the thread profile.

3. Orthodontic anchorage screw (1, 1′, 1″) according to claim 1, in which the thread pitch is designed, viewed along the longitudinal axis of the threaded barrel (4), in a non-constant manner.

4. Orthodontic anchorage screw (1, 1′, 1″) according to claim 1, the cross-section of whose threaded barrel (4) is approximately of triangular or trioval shape.

5. Orthodontic anchorage screw (1, 1′, 1″) according to any of claims 1, the outer contour of whose screw head (2) is designed with a polygonal contour, preferably with an octagonal contour.

6. Orthodontic anchorage screw (1, 1′, 1″) according to any of claims 1, the roof surface of whose screw head (2) is provided with a number of continuous profiled recesses (20), preferably with two crossing profiled recesses (20).

7. Orthodontic anchorage screw (1, 1′, 1″) according to claim 6, the cross-section and/or dimensioning of the, or each, profiled recess (20) being adapted to a dental therapeutical brace.

8. Orthodontic anchorage screw (1, 1′, 1″) according to claim 6, the side face of whose screw head (2) is provided with a peripheral fastening groove.

9. Orthodontic anchorage screw (1, 1′, 1″) according to any of claims 1, made of titanium as the basic material.
